SEQUENCE LISTING (63 sequences)

SEQ ID NO: 1 - DNA, 27 bp, Artificial Sequence
  Synthetic Construct
atgccctacctgatcgacttcggcctg

SEQ ID NO: 2 - protein, 9 aa, Artificial Sequence
  Synthetic Construct
MPYLIDFGL

SEQ ID NO: 3 - DNA, 99 bp, Artificial Sequence
  Synthetic Construct
ctgccaggacaggagatcgacaaggccgattcttacatgccctacctgatcgacttcggcctgagctccaagagcccatactctagcgtgaagaacccc

SEQ ID NO: 4 - protein, 33 aa, Artificial Sequence
  Synthetic Construct
LPGQEIDKADSYMPYLIDFGLSSKSPYSSVKNP

SEQ ID NO: 5 - DNA, 24 bp, Artificial Sequence
  Synthetic Construct
ggcgctgtgcagaacgaggtgacc

SEQ ID NO: 6 - protein, 9 aa, Artificial Sequence
  Synthetic Construct
GAVQNEVTL

SEQ ID NO: 7 - DNA, 99 bp, Artificial Sequence
  Synthetic Construct
accctgcacggacccacacctctgctgtacagactgggcgctgtgcagaacgaggtgaccctgacacaccctatcaccaagtacatcatggcctgcatg

SEQ ID NO: 8 - protein, 33 aa, Artificial Sequence
  Synthetic Construct
TLHGPTPLLYRLGAVQNEVTLTHPITKYIMACM

SEQ ID NO: 9 - DNA, 30 bp, Artificial Sequence
  Synthetic Construct
agaggcccaggaagagctttcgtgacaatc

SEQ ID NO: 10 - protein, 10 aa, Artificial Sequence
  Synthetic Construct
RGPGRAFVTI

SEQ ID NO: 11 - DNA, 99 bp, Artificial Sequence
  Synthetic Construct
cctaacaacaacaccagaaagtccatccggatcaggagaggcccaggaagagctttcgtgacaatcggcaagatcggaaacatgaggcaggctcactgt

SEQ ID NO: 12 - protein, 33 aa, Artificial Sequence
  Synthetic Construct
PNNNTRKSIRIRRGPGRAFVTIGKIGNMRQAHC

SEQ ID NO: 13 - DNA, 30 bp, Artificial Sequence
  Synthetic Construct
cgcggcccaggaagggcttttgtgaccatt

SEQ ID NO: 14 - DNA, 99 bp, Artificial Sequence
  Synthetic Construct
aacaacaacaccaggaagagaatccggatccagcgcggcccaggaagggcttttgtgaccattggcaagattggcaacatgaggcaggcccactgcaac

SEQ ID NO: 15 - protein, 33 aa, Artificial Sequence
  Synthetic Construct
NNNTRKRIRIQRGPGRAFVTIGKIGNMRQAHCN

SEQ ID NO: 16 - DNA, 24 bp, Artificial Sequence
  Synthetic Construct
acagtgtacggattctgtctgctg

SEQ ID NO: 17 - protein, 8 aa, Artificial Sequence
  Synthetic Construct
TVYGFCLL

SEQ ID NO: 18 - DNA, 99 bp, Artificial Sequence
  Synthetic Construct
gaggctagcaccgtggtgctggtgggagctaggggaacagtgtacggattctgtctgctgtccaacgacgtgtttagaatcgccgaggatatctctgtg

SEQ ID NO: 19 - protein, 33 aa, Artificial Sequence
  Synthetic Construct
EASTVVLVGARGTVYGFCLLSNDVFRIAEDISV

SEQ ID NO: 20 - DNA, 27 bp, Artificial Sequence
  Synthetic Construct
gcttccaacgagaacatggacgccatg

SEQ ID NO: 21 - protein, 9 aa, Artificial Sequence
  Synthetic Construct
ASNENMDAM

SEQ ID NO: 22 - DNA, 99 bp, Artificial Sequence
  Synthetic Construct
cccagaggcaagctgtccacccggggagtgcagatcgcttccaacgagaacatggacgccatggagtcctctacactggagctgcgctctaggtactgg

SEQ ID NO: 23 - protein, 33 aa, Artificial Sequence
  Synthetic Construct
PRGKLSTRGVQIASNENMDAMESSTLELRSRYW

SEQ ID NO: 24 - DNA, 27 bp, Artificial Sequence
  Synthetic Construct
gctatgcagatgctgaaggagaccatc

SEQ ID NO: 25 - protein, 9 aa, Artificial Sequence
  Synthetic Construct
AMQMLKETI

SEQ ID NO: 26 - DNA, 99 bp, Artificial Sequence
  Synthetic Construct
aacaccatgctgaacacagtgggcggacaccaggccgctatgcagatgctgaaggagaccatcaacgaggaggccgctgagtgggatagactgcaccca

SEQ ID NO: 27 - protein, 33 aa, Artificial Sequence
  Synthetic Construct
NTMLNTVGGHQAAMQMLKETINEEAAEWDRLHP

SEQ ID NO: 28 - DNA, 24 bp, Artificial Sequence
  Synthetic Construct
gtgtggctgagcgtgatctggatg

SEQ ID NO: 29 - protein, 8 aa, Artificial Sequence
  Synthetic Construct
VWLSVIWM

SEQ ID NO: 30 - DNA, 99 bp, Artificial Sequence
  Synthetic Construct
tccttcgtgcagtggtttgtgggcctgtctcccacagtgtggctgagcgtgatctggatgatgtggtactggggaccttccgtgtactctatcctgagc

SEQ ID NO: 31 - protein, 33 aa, Artificial Sequence
  Synthetic Construct
SFVQWFVGLSPTVWLSVIWMMWYWGPSVYSILS

SEQ ID NO: 32 - DNA, 27 bp, Artificial Sequence
  Synthetic Construct
tccccatcttacgtgtaccaccagttc

SEQ ID NO: 33 - protein, 9 aa, Artificial Sequence
  Synthetic Construct
SPSYVYHQF

SEQ ID NO: 34 - DNA, 99 bp, Artificial Sequence
  Synthetic Construct
gtgctggtggagctgtggcctcgggtgacctaccactccccatcttacgtgtaccaccagttcgagcggcgcgctaagtacaagagggagcccgtgagc

SEQ ID NO: 35 - protein, 33 aa, Artificial Sequence
  Synthetic Construct
VLVELWPRVTYHSPSYVYHQFERRAKYKREPVS

SEQ ID NO: 36 - DNA, 24 bp, Artificial Sequence
  Synthetic Construct
tctagcatcgagttcgctcgcctg

SEQ ID NO: 37 - protein, 8 aa, Artificial Sequence
  Synthetic Construct
SSIEFARL

SEQ ID NO: 38 - DNA, 99 bp, Artificial Sequence
  Synthetic Construct
ccttccgctaacgcctccgtggagaggatcaagaccacatctagcatcgagttcgctcgcctgcagtttacatacaaccacatccagaggcacgtgaac

SEQ ID NO: 39 - protein, 33 aa, Artificial Sequence
  Synthetic Construct
PSANASVERIKTTSSIEFARLQFTYNHIQRHVN

SEQ ID NO: 40 - DNA, 27 bp, Artificial Sequence
  Synthetic Construct
tcctacatcggatctatcaacaacatc

SEQ ID NO: 41 - protein, 9 aa, Artificial Sequence
  Synthetic Construct
SYIGSINNI

SEQ ID NO: 42 - DNA, 99 bp, Artificial Sequence
  Synthetic Construct
gaggagtacgctctgggcatcgtgggagtgctggagtcctacatcggatctatcaacaacatcaccaagcagtccgcttgcgtggccatgtctaagctg

SEQ ID NO: 43 - protein, 33 aa, Artificial Sequence
  Synthetic Construct
EEYALGIVGVLESYIGSINNITKQSACVAMSKL

SEQ ID NO: 44 - DNA, 39 bp, Artificial Sequence
  Synthetic Construct
gccaagtttgtggccgcttggacactgaaggccgctgcc

SEQ ID NO: 45 - protein, 13 aa, Artificial Sequence
  Synthetic Construct
AKFVAAWTLKAAA

SEQ ID NO: 46 - DNA, 21 bp, Artificial Sequence
  Synthetic Construct
aggggcaggaagaggagatcc

SEQ ID NO: 47 - DNA, 21 bp, Artificial Sequence
  Synthetic Construct
aggggaagaaagcggcgcagc

SEQ ID NO: 48 - DNA, 21 bp, Artificial Sequence
  Synthetic Construct
aggggcaggaagcggcgcagc

SEQ ID NO: 49 - DNA, 21 bp, Artificial Sequence
  Synthetic Construct
cgcggaaggaagaggagatct

SEQ ID NO: 50 - DNA, 21 bp, Artificial Sequence
  Synthetic Construct
agaggccggaagcggcgcagc

SEQ ID NO: 51 - DNA, 21 bp, Artificial Sequence
  Synthetic Construct
agaggccggaagaggagaagc

SEQ ID NO: 52 - DNA, 21 bp, Artificial Sequence
  Synthetic Construct
cgcggaaggaagcggcgcagc

SEQ ID NO: 53 - DNA, 21 bp, Artificial Sequence
  Synthetic Construct
agaggccggaaaaggagaagc

SEQ ID NO: 54 - DNA, 21 bp, Artificial Sequence
  Synthetic Construct
cgcggcaggaagaggagaagc

SEQ ID NO: 55 - DNA, 21 bp, Artificial Sequence
  Synthetic Construct
agaggccggaaacggcgctcc

SEQ ID NO: 56 - DNA, 21 bp, Artificial Sequence
  Synthetic Construct
cgcggcaggaaaaggagatct

SEQ ID NO: 57 - protein, 7 aa, Artificial Sequence
  Synthetic Construct
RGRKRRS

SEQ ID NO: 58 - DNA, 1362 bp, Artificial Sequence
  Synthetic Construct
atgctgccaggacaggagatcgacaaggccgattcttacatgccctacctgatcgacttcggcctgagctccaagagcccatactctagcgtgaagaaccccaggggcaggaagaggagatccaccctgcacggacccacacctctgctgtacagactgggcgctgtgcagaacgaggtgaccctgacacaccctatcaccaagtacatcatggcctgcatgaggggaagaaagcggcgcagccctaacaacaacaccagaaagtccatccggatcaggagaggcccaggaagagctttcgtgacaatcggcaagatcggaaacatgaggcaggctcactgtaggggcaggaagcggcgcagcaacaacaacaccaggaagagaatccggatccagcgcggcccaggaagggcttttgtgaccattggcaagattggcaacatgaggcaggcccactgcaaccgcggaaggaagaggagatctgaggctagcaccgtggtgctggtgggagctaggggaacagtgtacggattctgtctgctgtccaacgacgtgtttagaatcgccgaggatatctctgtgagaggccggaagcggcgcagccccagaggcaagctgtccacccggggagtgcagatcgcttccaacgagaacatggacgccatggagtcctctacactggagctgcgctctaggtactggagaggccggaagaggagaagcaacaccatgctgaacacagtgggcggacaccaggccgctatgcagatgctgaaggagaccatcaacgaggaggccgctgagtgggatagactgcacccacgcggaaggaagcggcgcagctccttcgtgcagtggtttgtgggcctgtctcccacagtgtggctgagcgtgatctggatgatgtggtactggggaccttccgtgtactctatcctgagcagaggccggaaaaggagaagcgtgctggtggagctgtggcctcgggtgacctaccactccccatcttacgtgtaccaccagttcgagcggcgcgctaagtacaagagggagcccgtgagccgcggcaggaagaggagaagcccttccgctaacgcctccgtggagaggatcaagaccacatctagcatcgagttcgctcgcctgcagtttacatacaaccacatccagaggcacgtgaacagaggccggaaacggcgctccgaggagtacgctctgggcatcgtgggagtgctggagtcctacatcggatctatcaacaacatcaccaagcagtccgcttgcgtggccatgtctaagctgcgcggcaggaaaaggagatctgccaagtttgtggccgcttggacactgaaggccgctgcc

SEQ ID NO: 59 - protein, 454 aa, Artificial Sequence
  Synthetic Construct
MLPGQEIDKADSYMPYLIDFGLSSKSPYSSVKNPRGRKRRSTLHGPTPLLYRLGAVQNEVTLTHPITKYIMACMRGRKRRSPNNNTRKSIRIRRGPGRAFVTIGKIGNMRQAHCRGRKRRSNNNTRKRIRIQRGPGRAFVTIGKIGNMRQAHCNRGRKRRSEASTVVLVGARGTVYGFCLLSNDVFRIAEDISVRGRKRRSPRGKLSTRGVQIASNENMDAMESSTLELRSRYWRGRKRRSNTMLNTVGGHQAAMQMLKETINEEAAEWDRLHPRGRKRRSSFVQWFVGLSPTVWLSVIWMMWYWGPSVYSILSRGRKRRSVLVELWPRVTYHSPSYVYHQFERRAKYKREPVSRGRKRRSPSANASVERIKTTSSIEFARLQFTYNHIQRHVNRGRKRRSEEYALGIVGVLESYIGSINNITKQSACVAMSKLRGRKRRSAKFVAAWTLKAAA

SEQ ID NO: 60 - DNA, 1302 bp, Artificial Sequence
  Synthetic Construct
atgctgccaggacaggagatcgacaaggccgattcttacatgccctacctgatcgacttcggcctgagctccaagagcccatactctagcgtgaagaaccccaggggcaggaagaggagatccaccctgcacggacccacacctctgctgtacagactgggcgctgtgcagaacgaggtgaccctgacacaccctatcaccaagtacatcatggcctgcatgaggggaagaaagcggcgcagccctaacaacaacaccagaaagtccatccggatcaggagaggcccaggaagagctttcgtgacaatcggcaagatcggaaacatgaggcaggctcactgtaggggcaggaagcggcgcagcaacaacaacaccaggaagagaatccggatccagcgcggcccaggaagggcttttgtgaccattggcaagattggcaacatgaggcaggcccactgcaaccgcggaaggaagaggagatctgaggctagcaccgtggtgctggtgggagctaggggaacagtgtacggattctgtctgctgtccaacgacgtgtttagaatcgccgaggatatctctgtgagaggccggaagcggcgcagccccagaggcaagctgtccacccggggagtgcagatcgcttccaacgagaacatggacgccatggagtcctctacactggagctgcgctctaggtactggagaggccggaagaggagaagcaacaccatgctgaacacagtgggcggacaccaggccgctatgcagatgctgaaggagaccatcaacgaggaggccgctgagtgggatagactgcacccacgcggaaggaagcggcgcagctccttcgtgcagtggtttgtgggcctgtctcccacagtgtggctgagcgtgatctggatgatgtggtactggggaccttccgtgtactctatcctgagcagaggccggaaaaggagaagcgtgctggtggagctgtggcctcgggtgacctaccactccccatcttacgtgtaccaccagttcgagcggcgcgctaagtacaagagggagcccgtgagccgcggcaggaagaggagaagcccttccgctaacgcctccgtggagaggatcaagaccacatctagcatcgagttcgctcgcctgcagtttacatacaaccacatccagaggcacgtgaacagaggccggaaacggcgctccgaggagtacgctctgggcatcgtgggagtgctggagtcctacatcggatctatcaacaacatcaccaagcagtccgcttgcgtggccatgtctaagctg

SEQ ID NO: 61 - protein, 434 aa, Artificial Sequence
  Synthetic Construct
MLPGQEIDKADSYMPYLIDFGLSSKSPYSSVKNPRGRKRRSTLHGPTPLLYRLGAVQNEVTLTHPITKYIMACMRGRKRRSPNNNTRKSIRIRRGPGRAFVTIGKIGNMRQAHCRGRKRRSNNNTRKRIRIQRGPGRAFVTIGKIGNMRQAHCNRGRKRRSEASTVVLVGARGTVYGFCLLSNDVFRIAEDISVRGRKRRSPRGKLSTRGVQIASNENMDAMESSTLELRSRYWRGRKRRSNTMLNTVGGHQAAMQMLKETINEEAAEWDRLHPRGRKRRSSFVQWFVGLSPTVWLSVIWMMWYWGPSVYSILSRGRKRRSVLVELWPRVTYHSPSYVYHQFERRAKYKREPVSRGRKRRSPSANASVERIKTTSSIEFARLQFTYNHIQRHVNRGRKRRSEEYALGIVGVLESYIGSINNITKQSACVAMSKL

SEQ ID NO: 62 - DNA, 24 bp, Artificial Sequence
  Synthetic Construct
ccctacctgatcgacttcggcctg

SEQ ID NO: 63 - protein, 8 aa, Artificial Sequence
  Synthetic Construct
PYLIDFGL

(19) United States
(12) Patent Application Publication
(10) Pub. No.: US 2024/0269267 A1
(43) Pub. Date: Aug. 15, 2024

(54) SYNTHETIC DNA VACCINE IMMUNOGENIC IMPROVEMENTS

(71) Applicant: The Wistar Institute of Anatomy and Biology, Philadelphia, PA (US)

(72) Inventors: David Weiner, Merion, PA (US); Pratik Bhojnagarwala, Philadelphia, PA (US); Ebony Gary, Philadelphia, PA (US)

(21) Appl. No.: 18/566,859

(22) PCT Filed: Jun. 3, 2022
(86) PCT No.: PCT/US2022/032138

§ 371 Date: Dec. 4, 2023

Related U.S. Application Data
(60) Provisional application No. 63/196,486, filed on Jun. 3, 2021.

(51) Int. Cl. A61K 39/295; A61K 39/00; A61P 31/16; A61P 31/22

(52) U.S. Cl. CPC A61K 39/295; A61P 31/16; A61P 31/22; A61K 2039/53; A61K 2039/572; C12N 2710/16134; C12N 2710/16634; C12N 2730/10134; C12N 2740/13034; C12N 2760/16134; C12N 2760/18534; C12N 2760/20234; C12N 2770/24234

(57) ABSTRACT

Disclosed herein is a composition comprising one or more viral antigen or a recombinant nucleic acid sequence that encodes one or more viral antigen with enhanced immunogenicity in vivo. Also disclosed herein is a method of generating an immune response in a subject by administering the composition to the subject. The disclosure also provides a method of preventing and/or treating a viral infection in a subject using said composition and methods.

### CROSS REFERENCE TO RELATED APPLICATIONS

This application claims priority to U.S. Provisional Patent Application No. 63/196,486, filed Jun. 3, 2021, which is hereby incorporated by reference herein in its entirety.

### TECHNICAL FIELD

The present invention relates to vaccines with improved immunogenicity comprising one or more recombinant nucleic acid sequence for generating one or more synthetic antigen in vivo, and a method of preventing and/or treating viral infection in a subject by administering said composition.

### BACKGROUND

Vaccination is an approach where antigenic materials are introduced into the hosts to elicit adaptive immune responses that may confer them with protection from subsequent pathogen exposure (Clem, 2011). Humoral immunity is an important branch of the adaptive immune system, in which antibodies produced by B cells serve either to directly neutralize targets on the pathogens through paratope-epitope interactions (Corti and Lanzavecchia, 2013; Kwong et al., 2013) or to indirectly mediate inactivation of the pathogens by engaging the complement system or effector cells such as macrophages and natural killer cells through Fc-dependent mechanisms (Kurdi et al., 2018; Seidel et al., 2013; van Erp et al., 2019). Antibody responses serve as an important correlate for protection for many emerging and re-emerging infectious diseases, including but not limited to HIV-1 (Burton and Hangartner, 2016), influenza (Laursen et al., 2018), and coronaviruses (Jiang et al., 2020). A strategy to enhance humoral responses induced by vaccination is, therefore, of great significance.

Direct incorporation and fusion of a potent CD4+ helper epitope with the target antigen may represent a simple and effective strategy to enhance the induced humoral immunity. Several important epitopes have been identified in this manner. Incorporation of Pan DR epitope (PADRE), for example, has demonstrated to improve immunogenicity of peptide and protein vaccines in animal studies, and it has also been explored in several clinical studies (Alexander et al., 2000; Ghaffari-Nazari et al., 2015: Snook et al., 2019). Identification of additional potent immunogenic motifs can create new tools to be used in conjunction with, or as alternative to, these established CD4-helper epitopes to increase responses induced by various vaccine antigens.

Thus, there is need in the art for nucleic acid-based vaccines with improved immunogenicity for the prevention and/or treatment of viral infection. The current invention satisfies this need.

### SUMMARY

In one embodiment, the present invention relates to a nucleic acid molecule comprising one or more nucleotide sequence encoding one or more antigen, wherein said one or more antigen comprises: a) an established T cell epitope: b) a series of amino acids N-terminal to said established T cell epitope derived from the endogenous protein from which the established T cell epitope originates; and c) a series of amino acids C-terminal to said established T cell epitope derived from the endogenous protein from which the established T cell epitope originates. In one embodiment, said one or more antigen is a viral antigen and said established T cell epitope is an established T cell viral epitope.

In one embodiment, said established T cell viral epitope of the nucleic acid molecule is derived from one or more virus selected from the group consisting of: Vesicular stomatitis virus (VSV), Hepatitis C virus (HCV), Human immunodeficiency virus 1 (HIV-1), Murine Cytomegalovirus (MCMV), Influenza A virus, Hepatitis B virus (HBV), Murine leukemia virus (MLV), Herpes simplex virus 1 (HSV-1), Herpes simplex virus 2 (HSV-2), and Respiratory syncytial virus (RSV).

In one embodiment, said established T cell viral epitope of the nucleic acid molecule comprises a peptide between 8 and 10 amino acids in length. In one embodiment, said established T cell viral epitope is encoded by one or more nucleotide sequence selected from the group consisting of: SEQ ID NO: 1, SEQ ID NO: 5, SEQ ID NO: 9, SEQ ID NO: 13, SEQ ID NO: 16, SEQ ID NO: 20, SEQ ID NO: 24, SEQ ID NO: 28, SEQ ID NO: 32, SEQ ID NO: 36, SEQ ID NO: 40 and SEQ ID NO: 62. In one embodiment, said established T cell viral epitope comprises an amino acid sequence that is one or more selected from the group consisting of: SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41 and SEQ ID NO: 63.

In one embodiment of the nucleic acid molecule of the present invention, the series of amino acids N-terminal to said established T cell epitope is between 11 and 13 amino acids in length. In one embodiment, the series of amino acids N-terminal to said established T cell epitope are at least 95% identical to a sequence N-terminal to the established T cell viral epitope in the endogenous protein from which the epitopes derives. In one embodiment, the sequence N-terminal to the established T cell viral epitope in the endogenous protein from which the epitopes derives is directly upstream of the established T cell epitope.

In one embodiment of the nucleic acid molecule of the present invention, the series of amino acids C-terminal to said established T cell epitope is between 11 and 13 amino acids in length. In one embodiment, the series of amino acids C-terminal to said established T cell epitope are at least 95% identical to a sequence C-terminal to the established T cell viral epitope in the endogenous protein from which the epitopes derives. In one embodiment, the sequence C-terminal to the established T cell viral epitope in the endogenous protein from which the epitopes derives is directly downstream of the established T cell epitope.

In one embodiment of the nucleic acid molecule of the present invention, said one or more viral antigen is encoded by one or more nucleotide sequence selected from the group consisting of: SEQ ID NO: 3, SEQ ID NO: 7, SEQ ID NO: 11, SEQ ID NO: 14, SEQ ID NO: 18, SEQ ID NO: 22, SEQ ID NO: 26, SEQ ID NO: 30, SEQ ID NO: 34, SEQ ID NO: 38, and SEQ ID NO: 42. In one embodiment, said one or more viral antigen comprises one or more amino acid sequence selected from the group consisting of: SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, and SEQ ID NO: 43.

In one embodiment, the nucleic acid molecule of the present invention comprises two or more nucleotide sequences encoding two or more antigens. In one embodiment, said two or more nucleotide sequences are separated by one or more nucleotide sequence encoding a furin cleavage domain (SEQ ID NO: 57). In one embodiment, the nucleic acid molecule of the present invention further comprises a nucleotide sequence encoding a Pan DR CD4+ helper epitope (SEQ ID NO: 45).

In one embodiment, the present invention relates to a method of administering an immunogenic composition to a subject in need thereof, the method comprising administering to said subject a nucleic acid molecule comprising one or more nucleotide sequence encoding one or more antigen, wherein said one or more antigen comprises: a) an established T cell epitope between 8 and 10 amino acids in length: b) a series of amino acids N-terminal to said established T cell epitope derived from the endogenous protein from which the established T cell epitope originates and between 11 and 13 amino acids in length; and c) a series of amino acids C-terminal to said established T cell epitope derived from the endogenous protein from which the established T cell epitope originates and between 11 and 13 amino acids in length.

In one embodiment, the present invention relates to a method of treating or protecting against viral infection in a subject in need thereof, the method comprising administering to said subject a nucleic acid molecule comprising one or more nucleotide sequence encoding one or more viral antigen, wherein said one or more antigen comprises: a) an established T cell viral epitope between 8 and 10 amino acids in length: b) a series of amino acids N-terminal to said established T cell viral epitope derived from the endogenous protein from which the established T cell viral epitope originates and between 11 and 13 amino acids in length; and c) a series of amino acids C-terminal to said established T cell viral epitope derived from the endogenous protein from which the established T cell viral epitope originates and between 11 and 13 amino acids in length. In one embodiment of the method, said established T cell viral epitope and said endogenous protein is derived from one or more virus selected from the group consisting of: VSV, HCV, HIV-1, MCMV, Influenza A virus, HBV, MLV, HSV-1, HSV-2, or RSV.

### BRIEF DESCRIPTION OF THE DRAWINGS

FIG. 1A and FIG. 1B depict the viral epitopes and the vaccination scheme used to assess immunogenicity. FIG. 1A depicts the virus, viral epitope, MHC molecule and mouse strain. FIG. 1B depicts a schematic of the vaccination scheme.

FIG. 2 depicts exemplary results demonstrating improved immunogenicity of incorporating endogenous flanking regions for each viral epitope. Splenocytes from vaccinated CBF1/J mice were assessed by ELISpot for the production of IFNγ in response to respective viral epitope peptides.

FIG. 3 depicts exemplary results demonstrating a greater number of immunogenic epitopes when incorporating endogenous flanking regions for each viral epitope.

### DETAILED DESCRIPTION

The present invention relates to compositions and methods for inducing an immune response against one or more antigen in a subject. In some embodiments, the present invention relates to sequential lineage antigens of that can be used as immunogens to induce a protective or therapeutic immune response in a subject. In some embodiments, the composition comprises compositions and methods for inducing an immune response against one or more viral antigen in a subject. In some embodiments, the composition comprises a protein or peptide comprising one or more of the viral antigens described herein. In some embodiments, the composition comprises a virus, including for example an inactivated virus or attenuated virus, expressing one or more of the antigens described herein. In some embodiments, the composition comprises a protein or peptide comprising one or more of the caner antigens described herein.

In some embodiments, the composition comprises a nucleic acid molecule, including for example, DNA, cDNA, RNA, and the like, encoding one or more of the viral antigens described herein. In embodiment, the one or more viral antigen comprises one or more established T cell viral epitope. In one embodiment, the one or more established T cell viral epitope is flanked N-terminally and C-terminally by additional amino acids from the respective endogenous viral protein.

In some embodiments, the present invention provides a composition comprising a nucleic acid molecule encoding one or more viral antigen, where the one or more viral antigen induces an immune response against one or more virus in the subject. In some embodiments, the induced immune response is an adaptive immune response. For example, in some embodiments, the composition comprises a vaccine comprising a nucleic acid molecule encoding one or more viral antigen. In some embodiments, the one or more viral antigen induces expression of a protective antibody. In some embodiments, the one or more viral antigen provides an adjuvant function.

In one embodiment, the composition of the invention comprises in vitro transcribed (IVT) RNA. For example, in some embodiments, the composition of the invention comprises IVT RNA which encodes one or more viral antigen, where the one or more viral antigen induces an adaptive immune response.

In some embodiments, the one or more viral antigen is based upon lineage immunogens that are able to initiate and mature an immune response against one or more rapidly mutating virus. In some embodiments, the one or more viral antigen is one selected for being maintained in the genome of the one or more virus, even while the one or more virus genome mutates to avoid immune surveillance.

In some embodiments, the antigen-encoding nucleic acid of the present composition is a nucleoside-modified RNA. In some embodiments, the antigen-encoding nucleic acid of the present composition is a purified nucleoside-modified RNA. For example, in some embodiments, the composition is purified such that it is free of double-stranded contaminants.

In some embodiments, the composition comprises a lipid nanoparticle (LNP). For example, in one embodiment, the composition comprises one or more viral antigen-encoding nucleic acid molecule encapsulated within a LNP. In some instances, the LNP enhances cellular uptake of the nucleic acid molecule.

In some embodiments, the composition comprises one or more immune modulator to enhance the immunogenic response. In one embodiment, the immune modulator is a CD4+ helper epitope. In one embodiment, the CD4+ helper epitope is Pan DR epitope (PADRE). In some embodiments, the immune modulator is an adjuvant. In some embodiments, the composition comprises a nucleic acid molecule encoding an adjuvant. For example, in one embodiment, the composition comprises a nucleoside-modified RNA encoding an adjuvant. In one embodiment, the composition comprises a nucleoside-modified RNA encoding one or more viral antigen and an adjuvant. In one embodiment, the composition comprises a first nucleoside-modified RNA, which encodes one or more viral antigen, and a second nucleoside-modified RNA, which encodes an adjuvant. In one embodiment, the composition comprises a nucleoside-modified RNA encoding an adjuvant and a LNP, wherein the LNP has adjuvant activity.

In one embodiment, the present invention provides a method for inducing an immune response against one or more virus in a subject. In some embodiments, the method comprises administering to the subject a composition comprising one or more nucleoside-modified RNA encoding one or more viral antigen, adjuvant, or a combination thereof.

In one embodiment, the method comprises the administration of the composition into the subject, including for example intradermal administration or intramuscular administration. In some embodiments, the method comprises administering a plurality of doses to the subject. In another embodiment, the method comprises administering a single dose of the composition, where the single dose is effective in inducing an adaptive immune response. In one embodiment, the method provides a sustained or prolonged immune response.

In some embodiments, the virus includes, but is not limited to, Vesicular stomatitis virus (VSV), Hepatitis C virus (HCV), Human immunodeficiency virus 1 (HIV-1), Murine Cytomegalovirus (MCMV), Influenza A virus, Hepatitis B virus (HBV), Murine leukemia virus (MLV), Herpes simplex virus 1 (HSV-1), Herpes simplex virus 2 (HSV-2), or Respiratory syncytial virus (RSV).

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary.” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane: their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° ° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° ° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof: or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Composition

The present invention relates to a composition comprising a recombinant nucleic acid sequence encoding one or more antigen, a fragment thereof, a variant thereof, or a combination thereof. In some embodiments, the composition comprises a recombinant nucleic acid sequence encoding one or more viral antigen, a fragment thereof, a variant thereof, or a combination thereof. The composition, when administered to a subject in need thereof, can result in the induction of an immune response to the one or more viral antigen in the subject.

In one embodiment, the composition comprises one or more nucleic acid molecule comprising one or more nucleotide sequence encoding an antigen. In one embodiment, the composition comprises one or more nucleic acid molecule comprising one or more nucleotide sequence encoding a viral antigen.

In one embodiment, the one or more nucleic acid molecule comprises one or more nucleotide sequence encoding a viral antigen from one or more virus selected from the group consisting of: Vesicular stomatitis virus (VSV), Hepatitis C virus (HCV), Human immunodeficiency virus 1 (HIV-1), Murine Cytomegalovirus (MCMV), Influenza A virus, Hepatitis B virus (HBV), Murine leukemia virus (MLV), Herpes simplex virus 1 (HSV-1), Herpes simplex virus 2 (HSV-2), or Respiratory syncytial virus (RSV).

In one embodiment, said one or more nucleic acid molecule comprises one or more nucleotide sequence encoding an established T cell epitope. In one embodiment, the one or more nucleotide sequence encoding an established T cell epitope encodes an amino acid sequence at least 1, at least 2, at least 3, at least 4, at least 5, at least 6, at least 7, at least 8, at least 9, at least 10, at least 11, at least 12, at least 13, at least 14, or at least 15 amino acids in length. In one embodiment, the one or more nucleotide sequence encoding an established T cell epitope encodes an amino acid sequence between 8 and 10 amino acids in length. In one embodiment, said established T cell epitope is an established T cell viral epitope.

In one embodiment, the one or more nucleotide sequence encoding an established T cell viral epitope encodes one or more selected from the group consisting of: VSV ML9, VSV PL8, HCV GL9, HIV RI10, MCMV TL8, Influenza A AM9, HIV AI9, HBV VM8, MLV SF9, HSV SL8, and RSV SI9. In one embodiment, the one or more nucleotide sequence encoding an established T cell viral epitope encodes one or more amino acid sequence at least 90%, at least 91%, at least 92%, at least 93%, at least 94%, at least 95%, at least 96%, at least 97%, at least 98%, or at least 99% identical to an amino acid sequence selected from the group consisting of: SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41 and SEQ ID NO: 63. In one embodiment, the one or more nucleotide sequence encoding an established T cell viral epitope encodes one or more amino acid sequence selected from the group consisting of: SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41 and SEQ ID NO: 63.

In one embodiment, the one or more nucleotide sequence encoding an established T cell viral epitope comprises one or more nucleic acid sequence at least 90%, at least 91%, at least 92%, at least 93%, at least 94%, at least 95%, at least 96%, at least 97%, at least 98%, or at least 99% identical to a nucleic acid sequence selected from the group consisting of: SEQ ID NO: 1, SEQ ID NO: 5, SEQ ID NO: 9, SEQ ID NO: 13, SEQ ID NO: 16, SEQ ID NO: 20, SEQ ID NO: 24, SEQ ID NO: 28, SEQ ID NO: 32, SEQ ID NO: 36, SEQ ID NO: 40 and SEQ ID NO: 62. In one embodiment, the one or more nucleotide sequence encoding an established T cell viral epitope comprises one or more nucleic acid sequence selected from the group consisting of: SEQ ID NO: 1, SEQ ID NO: 5, SEQ ID NO: 9, SEQ ID NO: 13, SEQ ID NO: 16, SEQ ID NO: 20, SEQ ID NO: 24, SEQ ID NO: 28, SEQ ID NO: 32, SEQ ID NO: 36, SEQ ID NO: 40 and SEQ ID NO: 62.

In one embodiment, said one or more nucleic acid molecule comprises one or more nucleotide sequence encoding one or more established T cell epitope that is flanked N-terminally and C-terminally by additional amino acids from the respective endogenous viral protein. In one embodiment, the number of additional N-terminal amino acids is at least 1, at least 2, at least 3, at least 4, at least 5, at least 6, at least 7, at least 8, at least 9, at least 10, at least 11, at least 12, at least 13, at least 14, or at least 15 amino acids. In one embodiment, the number of additional N-terminal amino acids is between 11 and 13 amino acids. In one embodiment, the number of additional C-terminal amino acids is at least 1, at least 2, at least 3, at least 4, at least 5, at least 6, at least 7, at least 8, at least 9, at least 10, at least 11, at least 12, at least 13, at least 14, or at least 15 amino acids. In one embodiment, the number of additional C-terminal amino acids is between 11 and 13 amino acids.

In one embodiment, said one or more nucleic acid molecule comprising one or more nucleotide sequence encodes one or more amino acid sequence at least 90%, at least 91%, at least 92%, at least 93%, at least 94%, at least 95%, at least 96%, at least 97%, at least 98%, or at least 99% identical to an amino acid sequence selected from the group consisting of: SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, and SEQ ID NO: 43. In one embodiment, said one or more nucleic acid molecule comprising one or more nucleotide sequence encodes one or more amino acid sequence selected from the group consisting of: SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, and SEQ ID NO: 43.

In one embodiment, said one or more nucleic acid molecule comprises one or more nucleic acid sequence at least 90%, at least 91%, at least 92%, at least 93%, at least 94%, at least 95%, at least 96%, at least 97%, at least 98%, or at least 99% identical to a nucleic acid sequence selected from the group consisting of: SEQ ID NO: 3, SEQ ID NO: 7, SEQ ID NO: 11, SEQ ID NO: 14, SEQ ID NO: 18, SEQ ID NO: 22, SEQ ID NO: 26, SEQ ID NO: 30, SEQ ID NO: 34, SEQ ID NO: 38, and SEQ ID NO: 42. In one embodiment, said one or more nucleic acid molecule comprises one or more nucleic acid sequence selected from the group consisting of: SEQ ID NO: 3, SEQ ID NO: 7, SEQ ID NO: 11, SEQ ID NO: 14, SEQ ID NO: 18, SEQ ID NO: 22, SEQ ID NO: 26, SEQ ID NO: 30, SEQ ID NO: 34, SEQ ID NO: 38, and SEQ ID NO: 42.

In one embodiment, said one or more nucleic acid molecule comprises one or more nucleotide sequence encoding a CD4+ helper epitope. In one embodiment, said CD4+ helper epitope comprises a Pan DR epitope (PADRE). In one embodiment, said PADRE comprises an amino acid sequence of SEQ ID NO: 45. In one embodiment, said PADRE is encoded by a nucleic acid sequence of SEQ ID NO: 46.

In one embodiment, said one or more nucleic acid molecule comprises a nucleotide sequence encoding a cleavage domain. In one embodiment, said cleavage domain is an enzymatic cleavage domain. In one embodiment, the enzymatic cleavage domain is a furin cleavage domain. In one embodiment, said nucleotide sequence encoding said furin cleavage domain comprises one or more nucleotide sequence selected from the group consisting of: SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56. In one embodiment, the nucleotide sequence encoding said furin cleavage domain encodes an amino acid sequence of SEQ ID NO: 57.

In one embodiment, the one or more nucleic acid molecule comprises a single nucleic acid molecule comprising at least 2, at least 3, at least 4, at least 5, at least 6, at least 7, at least 8, at least 9, at least 10, or at least 11 nucleotide sequences encoding an antigen. In one embodiment, the one or more nucleic acid molecule comprises a single nucleic acid molecule comprising at least 2, at least 3, at least 4, at least 5, at least 6, at least 7, at least 8, at least 9, at least 10, or at least 11 nucleotide sequences encoding a viral antigen. In one embodiment, each nucleotide sequence encoding a viral antigen is separated by nucleotide sequence encoding a cleavage domain. In one embodiment, the cleavage domain is furin. In one embodiment, said single nucleic acid molecule comprises a nucleic acid sequence of SEQ ID NO: 60. In one embodiment, said single nucleic acid molecule encodes an amino acid sequence of SEQ ID NO: 61. In one embodiment, said single nucleic acid molecule further comprises a nucleotide sequence encoding a CD4+ helper epitope. In one embodiment, said CD4+ helper epitope comprises a Pan DR epitope (PADRE). In one embodiment, said single nucleic acid molecule further comprising a nucleotide sequence encoding a CD4+ helper epitope comprises a nucleic acid sequence of SEQ ID NO: 58. In one embodiment, said single nucleic acid molecule further comprising a nucleotide sequence encoding a CD4+ helper epitope is encoded by an amino acid sequence of SEQ ID NO: 59.

The composition of the invention can treat, prevent and/or protect against any disease, disorder, or condition associated with a foreign or aberrantly expressed antigen in a subject. The composition of the invention can treat, prevent and/or protect against any disease, disorder, or condition associated with a viral infection in a subject. In certain embodiments, the composition can treat, prevent, and or/protect against VSV, HCV, HIV-1, MCMV, Influenza A virus, HBV, MLV, HSV-1, HSV-2, or RSV infection, or any combination thereof. In certain embodiments, the composition can treat, prevent, and or/protect against a condition associated with VSV, HCV, HIV-1, MCMV, Influenza A virus, HBV, MLV, HSV-1, HSV-2, or RSV infection, or any combination thereof.

The composition can result in the generation of the endogenous antibody against the antigen in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the antigen in the subject within at least about 1 day, 2 days, 3 days, 4 days, 5 days, 6 days, 7 days, 8 days, 9 days, or 10 days of administration of the composition to the subject. The composition can result in generation of the antigen in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of more endogenous antibody in the subject than the generation of an endogenous antibody in a subject who is administered a composition comprising a single viral antigen to induce a humoral immune response.

The composition, when administered to the subject in need thereof, can result in the generation of more endogenous antibody in the subject than the generation of an endogenous antibody in a subject who is administered a composition comprising nucleic acid encoding a single viral antigen to induce a humoral immune response.

The composition, when administered to the subject in need thereof, can result in the generation of more endogenous antibody in the subject than the generation of an endogenous antibody in a subject who is administered a composition comprising a single viral antigen comprising an established T cell epitope to induce a humoral immune response.

The composition, when administered to the subject in need thereof, can result in the generation of more endogenous antibody in the subject than the generation of an endogenous antibody in a subject who is administered a composition comprising nucleic acid encoding a single antigen comprising an established T cell epitope to induce a humoral immune response.

The composition of the present invention can have features required of effective compositions such as being safe so that the composition does not cause illness or death: being protective against illness; and providing ease of administration, few side effects, biological stability and low cost per dose.

### 3. Recombinant Nucleic Acid Sequence

As described above, the composition can comprise a recombinant nucleic acid sequence. The recombinant nucleic acid sequence can encode one or more antigen, a fragment thereof, a variant thereof, or a combination thereof. The recombinant nucleic acid sequence can encode one or more viral antigen, a fragment thereof, a variant thereof, or a combination thereof. The viral antigens are described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription: mRNA stability and codon optimization: addition of a kozak sequence (e.g., GCC ACC) for increased translation: addition of an immunoglobulin (Ig) leader sequence encoding a signal peptide: addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or a eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences.

### (1) Viral Antigens

The recombinant nucleic acid sequence construct can include one or more nucleic acid sequence encoding one or more viral antigen, a fragment thereof, a variant thereof, or a combination thereof. The one or more viral antigen can include an established T cell viral epitope, an amino acid sequence N-terminal to said established T cell viral epitope that is derived from the respective endogenous viral protein, an amino acid sequence C-terminal to said established T cell viral epitope that is derived from the respective endogenous viral protein, or a combination thereof.

In some embodiments, the established T cell viral epitope is 8-10 amino acids in length. In some embodiments, the viral epitope is one or more selected from the group consisting of: VSV ML9, VSV PL8, HCV GL9, HIV RI10, MCMV TL8, Influenza A AM9, HIV AI9, HBV VM8, MLV SF9, HSV SL8, and RSV SI9.

In some embodiments, the amino acid sequence N-terminal to said established T cell viral epitope is 11-13 amino acids in length. In some embodiments, the amino acid sequence N-terminal to said established T cell viral epitope is at least 95%, at least 96%, at least 97%, at least 98%, or at least 99% identical to a sequence N-terminal to the established T cell viral epitope in the endogenous viral protein. In some embodiments, the amino acid sequence N-terminal to said established T cell viral epitope comprises a sequence N-terminal to the established T cell viral epitope in the endogenous viral protein. In some embodiments, the sequence N-terminal to the established T cell viral epitope in the endogenous viral protein is directly upstream of the established T cell viral epitope.

In some embodiments, the amino acid sequence C-terminal to said established T cell viral epitope is 11-13 amino acids in length. In some embodiments, the amino acid sequence C-terminal to said established T cell viral epitope is at least 95%, at least 96%, at least 97%, at least 98%, or at least 99% identical to a sequence C-terminal to the established T cell viral epitope in the endogenous viral protein. In some embodiments, the amino acid sequence C-terminal to said established T cell viral epitope comprises a sequence C-terminal to the established T cell viral epitope in the endogenous viral protein. In some embodiments, the sequence C-terminal to the established T cell viral epitope in the endogenous viral protein is directly downstream of the established T cell viral epitope.

### (2) CD4+ Helper Epitope

The recombinant nucleic acid sequence construct can include one or more nucleic acid sequence encoding a CD4+ helper epitope. CD4+ helper epitopes enhance the induced humoral immunity of a desired antigen. In one embodiment, the CD4+ helper epitope comprises a Pan DR epitope (PADRE).

### (3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another.

In one embodiment, at least 1, at least 2, at least 3, at least 4, at least 5, at least 6, at least 7, at least 8, at least 9, at least 10, or at least 11 recombinant nucleic acid sequence constructs can include a nucleic acid sequence encoding at least 1, at least 2, at least 3, at least 4, at least 5, at least 6, at least 7, at least 8, at least 9, at least 10, or at least 11 antigens, respectively. In one embodiment, at least 1, at least 2, at least 3, at least 4, at least 5, at least 6, at least 7, at least 8, at least 9, at least 10, or at least 11 recombinant nucleic acid sequence constructs can include a nucleic acid sequence encoding at least 1, at least 2, at least 3, at least 4, at least 5, at least 6, at least 7, at least 8, at least 9, at least 10, or at least 11 viral antigens, respectively. In one embodiment, an additional recombinant nucleic acid sequence comprises a nucleic acid sequence encoding a CD4+ helper epitope. In some embodiments, each individual recombinant nucleic acid sequence construct can be placed in a separate vector.

In one embodiment, a single recombinant nucleic acid sequence construct can include one or more nucleic acid sequence encoding at least 1, at least 2, at least 3, at least 4, at least 5, at least 6, at least 7, at least 8, at least 9, at least 10, or at least 11 antigens. In one embodiment, a single recombinant nucleic acid sequence construct can include one or more nucleic acid sequence encoding at least 1, at least 2, at least 3, at least 4, at least 5, at least 6, at least 7, at least 8, at least 9, at least 10, or at least 11 viral antigens. In some embodiments, each nucleotide sequence encoding a viral antigen is separated by a protease cleavage site, as previously described. In one embodiment, the recombinant nucleic acid sequence further comprises a nucleic acid sequence encoding a CD4+ helper epitope. In some embodiments, said nucleic acid sequence encoding a CD4+ helper epitope is 5′ to the first viral antigen and further separated by a nucleic acid sequence encoding a protease cleavage site. In some embodiments, said nucleic acid sequence encoding a CD4+ helper epitope is 3′ to the last viral antigen and further separated by a nucleic acid sequence encoding a protease cleavage site. In some embodiments, the single recombinant nucleic acid sequence construct can be placed in a single vector.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (12) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (13) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### (14) RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90%, at least 91%, at least 92%, at least 93%, at least 94%, at least 95%, at least 96%, at least 97%, at least 98%, or at least 99% homologous to one of SEQ ID NO: 1, SEQ ID NO: 5, SEQ ID NO: 9, SEQ ID NO: 13, SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 62, SEQ ID NO: 3, SEQ ID NO: 7, SEQ ID NO: 11, SEQ ID NO: 14, SEQ ID NO: 18, SEQ ID NO: 22, SEQ ID NO: 26, SEQ ID NO: 30, SEQ ID NO: 34, SEQ ID NO: 38, and SEQ ID NO: 42. In another embodiment, the nucleotide sequence comprises an RNA sequence transcribed by a DNA sequence encoding a polypeptide sequence of SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 63, SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, or a variant thereof or a fragment thereof. Accordingly, in one embodiment, the invention provides an RNA molecule encoding one or more of the viral antigens. The RNA may be plus-stranded. Accordingly, in some embodiments, the RNA molecule can be translated by cells without needing any intervening replication steps such as reverse transcription. An RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### (15) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### (16) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584: WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (17) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 4. Antigens

The present invention also relates to a composition comprising one or more antigen capable of inducing a robust immune response in a subject. In one embodiment, the composition comprises one or more viral antigen. One of skill in the art will recognize that the compositions and methods of the present invention can be useful in the production of an improved vaccine to boost the immunogenicity of any antigen, viral or otherwise.

### Viral Antigens

In one embodiment, the one or more viral antigen is associated with one or more infectious virus. In one embodiment, the one or more viral antigen is associated with one or more infectious virus.

In one embodiment, the antigen can be associated with a virus including, but not limited to, VSV, HCV, HIV-1, MCMV, Influenza A virus, HBV, MLV, HSV-1, HSV-2, or RSV. In one embodiment, the one or more viral antigen can be associated with viral infection. In one embodiment, the viral infection includes, but is not limited to, VSV, HCV, HIV-1, MCMV, Influenza A virus, HBV, MLV, HSV-1, HSV-2, or RSV infection.

In one embodiment, the antigen is an established T cell viral epitope of one or more virus including, but not limited to, VSV, HCV, HIV-1, MCMV, Influenza A virus, HBV, MLV, HSV-1, HSV-2, or RSV. In one embodiment, the established T cell viral epitope includes, but is not limited to, VSV ML9, VSV PL8, HCV GL9, HIV RI10, MCMV TL8, Influenza A AM9, HIV AI9, HBV VM8, MLV SF9, HSV SL8, and RSV SI9.

In one embodiment, the established T cell viral epitope comprises an amino acid sequence at least 90%, at least 91%, at least 92%, at least 93%, at least 94%, at least 95%, at least 96%, at least 97%, at least 98%, or at least 99% identical to an amino acid sequence of SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41, or SEQ ID NO: 63. In one embodiment, the established T cell viral epitope comprises an amino acid sequence of SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41 or SEQ ID NO: 63.

In one embodiment, the antigen is an established T cell viral epitope that is flanked N-terminally and C-terminally by additional amino acids from the respective endogenous viral protein. In one embodiment, the antigen comprises an amino acid sequence at least 90%, at least 91%, at least 92%, at least 93%, at least 94%, at least 95%, at least 96%, at least 97%, at least 98%, or at least 99% identical to an amino acid sequence of SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, or SEQ ID NO: 43. In one embodiment, the antigen comprises an amino acid sequence of SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, or SEQ ID NO: 43.

### Cancer Antigens

In some embodiments, the composition comprises one or more cancer antigen capable of inducing a robust immune response to cancerous cells or tissues. In some embodiments, the cancer antigen is derived from, but is not limited to, hTERT, PSA antigen, PSMA antigen, STEAP antigen, PSCA antigen, Prostatic acid phosphatase (PAP) antigen, Wilm's tumor suppressor gene 1 (WT1), Tyrosinase (Tyr), NY-ESO-1, Melanoma antigen preferentially expressed in tumors (PRAME), Melanoma-associated Antigen (MAGE: e.g. MAGE-A4), an immunogenic fragment, or an immunogenic variant thereof.

hTERT is a human telomerase reverse transcriptase that synthesizes a TTAGGG tag on the end of telomeres to prevent cell death due to chromosomal shortening. Hyperproliferative cells with abnormally high expression of hTERT may be targeted by immunotherapy. Recent studies demonstrate that hTERT expression in dendritic cells transfected with hTERT genes can induce CD8+ cytotoxic T cells and elicit a CD4+ T cells in an antigen-specific fashion.

The following are antigens capable of eliciting an immune response in a mammal against a prostate antigen. The consensus antigen can comprise epitopes that make them particularly effective as immunogens against prostate cancer cells can be induced. The consensus prostate antigen can comprise the full length translation product, a variant thereof, a fragment thereof or a combination thereof.

The prostate antigens can include one or more of the following: PSA antigen, PSMA antigen, STEAP antigen, PSCA antigen, Prostatic acid phosphatase (PAP) antigen, and other known prostate cancer markers. Proteins may comprise sequences homologous to the prostate antigens, fragments of the prostate antigens and proteins with sequences homologous to fragments of the prostate antigens.

WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug.

Mutation of WT1 can lead to tumor or cancer formation, for example, Wilm's tumor or tumors expressing WT1. Wilm's tumor often forms in one or both kidneys before metastasizing to other tissues, for example, but not limited to, liver tissue, urinary tract system tissue, lymph tissue, and lung tissue. Accordingly, Wilm's tumor can be considered a metastatic tumor. Wilm's tumor usually occurs in younger children (e.g., less than 5 years old) and in both sporadic and hereditary forms. Accordingly, the vaccine can be used for treating subjects suffering from Wilm's tumor. The vaccine can also be used for treating subjects with cancers or tumors that express WT1 for preventing development of such tumors in subjects. The WT1 antigen can differ from the native, “normal” WT1 gene, and thus, provide therapy or prophylaxis against an WT1 antigen-expressing tumor. Proteins may comprise sequences homologous to the WT1 antigens, fragments of the WT1 antigens and proteins with sequences homologous to fragments of the WT1 antigens.

The Tyrosinase (Tyr) antigen is an important target for immune mediated clearance by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8⁺ (CTL) to attack and kill tumor cells; (3) increase T helper cell responses; (4) and increase inflammatory responses via IFN-γ and TFN-α or preferably all of the aforementioned.

Tyrosinase is a copper-containing enzyme that can be found in plant and animal tissues. Tyrosinase catalyzes the production of melanin and other pigments by the oxidation of phenols such as tyrosine. In melanoma, tyrosinase can become unregulated, resulting in increased melanin synthesis. Tyrosinase is also a target of cytotoxic T cell recognition in subjects suffering from melanoma. Accordingly, tyrosinase can be an antigen associated with melanoma.

The antigen can comprise protein epitopes that make them particularly effective as immunogens against which anti-Tyr immune responses can be induced. The Tyr antigen can comprise the full length translation product, a variant thereof, a fragment thereof or a combination thereof.

The Tyr antigen can comprise a consensus protein. The Tyr antigen induces antigen-specific T-cell and high titer antibody responses both systemically against all cancer and tumor related cells. As such, a protective immune response is provided against tumor formation by vaccines comprising the Tyr consensus antigen. Accordingly, any user can design a vaccine of the present invention to include a Tyr antigen to provide broad immunity against tumor formation, metastasis of tumors, and tumor growth. Proteins may comprise sequences homologous to the Tyr antigens, fragments of the Tyr antigens and proteins with sequences homologous to fragments of the Tyr antigens.

NY-ESO-1 is a cancer-testis antigen expressed in various cancers where it can induce both cellular and humoral immunity. Gene expression studies have shown upregulation of the gene for NY-ESO-1, CTAG1B, in myxoid and round cell liposarcomas. Proteins may comprise sequences homologous to the NYES01 antigens, fragments of the NYES01 antigens and proteins with sequences homologous to fragments of the NYES01 antigens.

Melanoma antigen preferentially expressed in tumors (PRAME antigen) is a protein that in humans is encoded by the PRAME gene. This gene encodes an antigen that is predominantly expressed in human melanomas and that is recognized by cytolytic T lymphocytes. It is not expressed in normal tissues, except testis. The gene is also expressed in acute leukemias. Five alternatively spliced transcript variants encoding the same protein have been observed for this gene. Proteins may comprise sequences homologous to the PRAME antigens, fragments of the PRAME antigens and proteins with sequences homologous to fragments of the PRAME antigens.

MAGE stands for Melanoma-associated Antigen, and in particular melanoma associated antigen 4 (MAGEA4). MAGE-A4 is expressed in male germ cells and tumor cells of various histological types such as gastrointestinal, esophageal and pulmonary carcinomas. MAGE-A4 binds the oncoprotein, Gankyrin. This MAGE-A4 specific binding is mediated by its C-terminus. Studies have shown that exogenous MAGE-A4 can partly inhibit the adhesion-independent growth of Gankyrin-overexpressing cells in vitro and suppress the formation of migrated tumors from these cells in nude mice. This inhibition is dependent upon binding between MAGE-A4 and Gankyrin, suggesting that interactions between Gankyrin and MAGE-A4 inhibit Gankyrin-mediated carcinogenesis. It is likely that MAGE expression in tumor tissue is not a cause, but a result of tumor genesis, and MAGE genes take part in the immune process by targeting early tumor cells for destruction.

Melanoma-associated antigen 4 protein (MAGEA4) can be involved in embryonic development and tumor transformation and/or progression. MAGEA4 is normally expressed in testes and placenta. MAGEA4, however, can be expressed in many different types of tumors, for example, melanoma, head and neck squamous cell carcinoma, lung carcinoma, and breast carcinoma. Accordingly, MAGEA4 can be antigen associated with a variety of tumors.

The MAGEA4 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune responses. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts.

The MAGEA4 antigen can comprise protein epitopes that make them particularly effective as immunogens against which anti-MAGEA4 immune responses can be induced. The MAGEA4 antigen can comprise the full length translation product, a variant thereof, a fragment thereof or a combination thereof. The MAGEA4 antigen can comprise a consensus protein.

The nucleic acid sequence encoding the consensus MAGEA4 antigen can be optimized with regards to codon usage and corresponding RNA transcripts. The nucleic acid encoding the consensus MAGEA4 antigen can be codon and RNA optimized for expression. In some embodiments, the nucleic acid sequence encoding the consensus MAGEA4 antigen can include a Kozak sequence (e.g., GCC ACC) to increase the efficiency of translation. The nucleic acid encoding the consensus MAGEA4 antigen can include multiple stop codons (e.g., TGA TGA) to increase the efficiency of translation termination.

### Tumor Antigens

In some embodiments, the composition comprises one or more tumor antigen capable of inducing a robust immune response to hyperproliferative cells or tissues, including cancerous tumors. In some embodiments, the tumor antigen is derived from, but is not limited to, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15: overexpressed embryonic antigens such as CEA: overexpressed oncogenes and mutated tumor-suppressor genes such as p53, Ras, HER-2/neu: unique tumor antigens resulting from chromosomal translocations: such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### 5. Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Serial No. 021,579 filed Apr. 1, 1994, which is fully incorporated by reference.

The composition may comprise DNA at quantities of from about 1 nanogram to 100 milligrams: about 1 microgram to about 10 milligrams: or preferably about 0.1 microgram to about 10 milligrams: or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams: from about 1 microgram to about 10 milligrams: from about 0.1 microgram to about 10 milligrams: from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 6. Method of Delivery of the Composition

The present invention also relates to a method of delivering the composition to the subject in need thereof. The method of delivery can include administering the composition to the subject. Administration can include, but is not limited to, DNA injection with and without in vivo electroporation, liposome mediated delivery, and nanoparticle facilitated delivery.

The subject receiving delivery of the composition may be a mammal. The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes: a hypodermic needle: an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 7. Method of Treatment

Also provided herein is a method of treating, protecting against, and/or preventing disease in a subject in need thereof by generating an immune response in the subject. The method can include administering the composition to the subject. Administration of the composition to the subject can be done using the method of delivery described above.

In certain embodiments, the invention provides a method of treating protecting against, and/or preventing on or more viral infection. In one embodiment, the method treats, protects against, and/or prevents a disease associated with one or more viral infection. In one embodiment, the virus includes, but is not limited to, VSV, HCV, HIV-1, MCMV, Influenza A virus, HBV, MLV, HSV-1, HSV-2, or RSV.

Upon expression of the one or more antigen and generation of endogenous antibodies against said one or more antigen in the subject, the antibodies can bind to or react with the one or more antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition comprising one or more viral antigen or comprising one or more nucleic acid encoding one or more viral antigen can treat, prevent, and/or protect against disease in the subject administered the composition. The compositions can promote survival of subject administered the composition. In one embodiment, the composition can provide increased survival of the subject over the expected survival of a subject having the disease who has not been administered the composition. In various embodiments, the composition can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of subjects administered the composition over the expected survival in the absence of the composition. In one embodiment, the composition can provide increased protection against the disease in the subject over the expected protection of a subject who has not been administered the composition. In various embodiments, the composition can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 8. Use in Combination

The present invention also provides a method of treating, protecting against, and/or preventing disease in a subject in need thereof by administering a combination of the composition of the present invention and a therapeutic agent. In one embodiment, the therapeutic agent is an antiviral agent.

The composition and a therapeutic agent may be administered using any suitable method such that a combination of the composition and therapeutic agent are both present in the subject. In one embodiment, the method may comprise administration of a first composition comprising one or more nucleic acid molecule or one or more antigen of the invention by any of the methods described in detail above and administration of a second composition comprising a therapeutic agent less than 1, less than 2, less than 3, less than 4, less than 5, less than 6, less than 7, less than 8, less than 9 or less than 10 days following administration of the first composition. In one embodiment, the method may comprise administration of a first composition comprising one or more nucleic acid molecule or one or more antigen of the invention by any of the methods described in detail above and administration of a second composition comprising a therapeutic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising a therapeutic agent and administration of a second composition comprising one or more nucleic acid molecule or one or more antigen of the invention by any of the methods described in detail above less than 1, less than 2, less than 3, less than 4, less than 5, less than 6, less than 7, less than 8, less than 9 or less than 10 days following administration of the therapeutic agent. In one embodiment, the method may comprise administration of a first composition comprising a therapeutic agent and administration of a second composition comprising one or more nucleic acid molecule or one or more antigen of the invention by any of the methods described in detail above more than 1, more than 2, more than 3, more than 4, more than 5, more than 6, more than 7, more than 8, more than 9 or more than 10 days following administration of the therapeutic agent. In one embodiment, the method may comprise administration of a first composition comprising one or more nucleic acid molecule or one or more antigen of the invention by any of the methods described in detail above and a second composition comprising a therapeutic agent concurrently. In one embodiment, the method may comprise administration of a first composition comprising one or more nucleic acid molecule or one or more antigen of the invention by any of the methods described in detail above and a second composition comprising a therapeutic agent concurrently. In one embodiment, the method may comprise administration of a single composition comprising one or more nucleic acid molecule or one or more antigen of the invention and a therapeutic agent.

Non-limiting examples of antiviral therapeutic agents that can be used in combination with the composition of the invention to protect against or treat viral infection include Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvada, Umifenovir, Valaciclovir (Valtrex), Valganciclovir (Valcyte), Vicriviroc, Vidarabine, Zalcitabine, Zanamivir (Relenza), or Zidovudine.

### 9. Generation of Antigens In Vitro and Ex Vivo

In one embodiment, the one or more antigen is generated in vitro or ex vivo. In one embodiment, the one or more viral antigen is generated in vitro or ex vivo. For example, in one embodiment, a nucleic acid encoding one or more viral antigen can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The present invention has multiple aspects, illustrated by the following non-limiting examples.

### 10. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

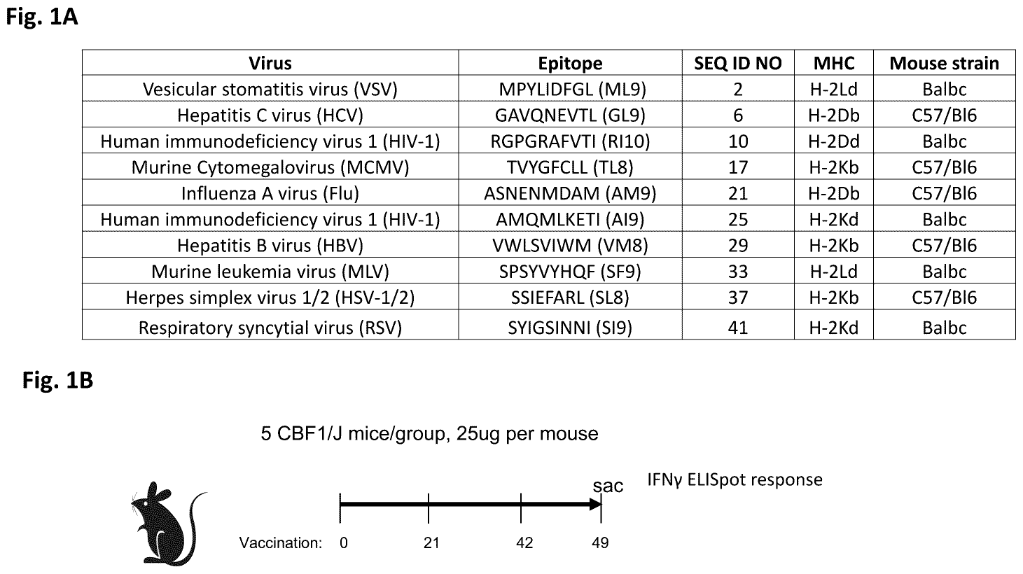

Various strategies have been employed to increase the immunogenicity of viral vaccines. Herein, various nucleic acid constructs encoding a variety of established T cell viral epitopes (8-11 amino acids in length: FIG. 1A) and one or more linker or immunogenic motif (see Table 1 below) were compared for immunogenicity in mice. Five CBF1/J mice were inoculated per group with 25 μg of nucleic acid per mouse. Forty-nine days after vaccination, the mice were sacrificed, splenocytes were isolated, and the IFNγ ELISpot was measured in response to individual viral epitope peptides (FIG. 1B).

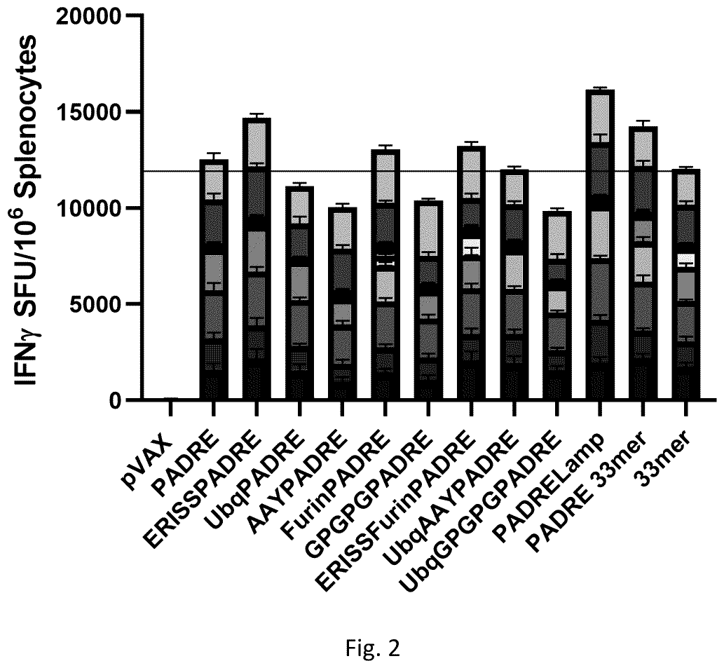

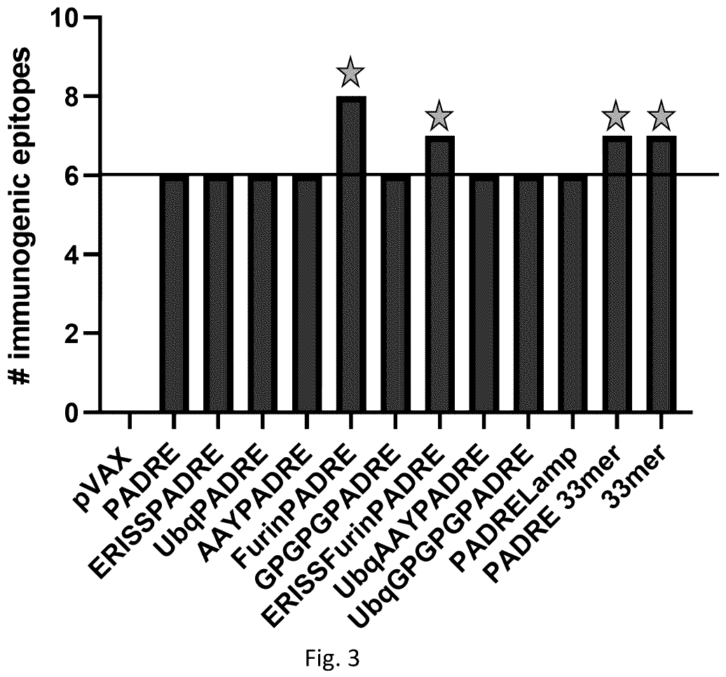

The results show that DNA encoded vaccines elicit strong immune responses against most epitopes (FIG. 2). Addition of AAY and GPGP linkers tends to generate weaker overall responses (FIG. 2), while the presence of furin cleavage sites generates immune responses against more epitopes (FIG. 3). Notably, a construct encoding the established T cell viral epitopes flanked N-terminally and C-terminally by 11-13 amino acids derived from the endogenous viral protein (33mer) and the same but further encoding the PADRE CD4+ helper epitope (PADRE 33mer) generated robust immune responses for most epitopes (FIG. 2) and generated immune responses against more epitopes than established T cell viral epitopes alone with PADRE (FIG. 3). Thus, the addition of N-terminal and C-terminal amino acid sequences that flank the established epitopes represents a novel strategy to improve the immunogenicity of nucleic acid vaccines.

TABLE 1
Vector constructs containing various
linkers and immunogenic motifs

| Group # | Vector Construct |
|---|---|
| 1 | pVax |
| 2 | Mouse Viral T cell epitopes_PADRE |
| 3 | Mouse Viral T cell epitopes_ERISSPADRE |
| 4 | Mouse Viral T cell epitopes_ubiquitinPADRE |
| 5 | Mouse Viral T cell epitopes_AAYPADRE |
| 6 | Mouse Viral T cell epitopes_FurinPADRE |
| 7 | Mouse Viral T cell epitopes_GPGPGPADRE |
| 8 | Mouse Viral T cell epitopes_ERISSFurinPADRE |
| 9 | Mouse Viral T cell epitopes_UbiqutinAAYPADRE |
| 10 | Mouse Viral T cell epitopes_UbiqutinGPGPGPADRE |
| 11 | Mouse Viral T cell epitopes_PADRELamp |
| 12 | Mouse Viral T cell epitopes_PADRE (33mers) |
| 13 | Mouse Viral T cell epitopes_33mers |

| SEQ | | | |
|---|---|---|---|
| ID | | | |
| NO: | NA/AA | Type | SEQUENCE |
| 1 | NA | VSV | ATGCCCTACCTGATCGACTTCGGCCTG |
| | | ML9 | |
| 2 | AA | VSV | MPYLIDFGL |
| | | ML9 | |
| 3 | NA | VSV | CTGCCAGGACAGGAGATCGACAAGGCCGATTCTTACATGCCCTACCT |
| | | ML9 | GATCGACTTCGGCCTGAGCTCCAAGAGCCCATACTCTAGCGTGAAGA |
| | | 33 mer | ACCCC |
| 4 | AA | VSV | LPGQEIDKADSYMPYLIDFGLSSKSPYSSVKNP |
| | | ML9 | |
| | | 33 mer | |
| 5 | NA | HCV | GGCGCTGTGCAGAACGAGGTGACC |
| | | GL9 | |
| 6 | AA | HCV | GAVQNEVTL |
| | | GL9 | |
| 7 | NA | HCV | ACCCTGCACGGACCCACACCTCTGCTGTACAGACTGGGCGCTGTGCA |
| | | GL9 | GAACGAGGTGACCCTGACACACCCTATCACCAAGTACATCATGGCCT |
| | | 33 mer | GCATG |
| 8 | AA | HCV | TLHGPTPLLYRLGAVQNEVTLTHPITKYIMACM |
| | | GL9 | |
| | | 33 mer | |
| 9 | NA | HIV | AGAGGCCCAGGAAGAGCTTTCGTGACAATC |
| | | RI10-1 | |
| 10 | AA | HIV RI10 | RGPGRAFVTI |
| 11 | NA | HIV | CCTAACAACAACACCAGAAAGTCCATCCGGATCAGGAGAGGCCCAG |
| | | RI10-1 | GAAGAGCTTTCGTGACAATCGGCAAGATCGGAAACATGAGGCAGGC |
| | | 33 mer | TCACTGT |
| 12 | AA | HIV | PNNNTRKSIRIRRGPGRAFVTIGKIGNMRQAHC |
| | | RI10-1 | |
| | | 33 mer | |
| 13 | NA | HIV | CGCGGCCCAGGAAGGGCTTTTGTGACCATT |
| | | RI10-2 | |
| 14 | NA | HIV | AACAACAACACCAGGAAGAGAATCCGGATCCAGCGCGGCCCAGGAA |
| | | RI10-2 | GGGCTTTTGTGACCATTGGCAAGATTGGCAACATGAGGCAGGCCCA |
| | | 33 mer | CTGCAAC |
| 15 | AA | HIV | NNNTRKRIRIQRGPGRAFVTIGKIGNMRQAHCN |
| | | RI10-2 | |
| | | 33 mer | |
| 16 | NA | MCMV | ACAGTGTACGGATTCTGTCTGCTG |
| | | TL8 | |
| 17 | AA | MCMV | TVYGFCLL |
| | | TL8 | |
| 18 | NA | MCMV | GAGGCTAGCACCGTGGTGCTGGTGGGAGCTAGGGGAACAGTGTAC |
| | | TL8 | GGATTCTGTCTGCTGTCCAACGACGTGTTTAGAATCGCCGAGGATAT |
| | | 33 mer | CTCTGTG |
| 19 | AA | MCMV | EASTVVLVGARGTVYGFCLLSNDVFRIAEDISV |
| | | TL8 | |
| | | 33 mer | |
| 20 | NA | Influenza | GCTTCCAACGAGAACATGGACGCCATG |
| | | AM9 | |
| 21 | AA | Influenza | ASNENMDAM |
| | | AM9 | |
| 22 | NA | Influenza | CCCAGAGGCAAGCTGTCCACCCGGGGAGTGCAGATCGCTTCCAACG |
| | | AM9 | AGAACATGGACGCCATGGAGTCCTCTACACTGGAGCTGCGCTCTAGG |
| | | 33 mer | TACTGG |
| 23 | AA | Influenza | PRGKLSTRGVQIASNENMDAMESSTLELRSRYW |
| | | AM9 | |
| | | 33 mer | |
| 24 | NA | HIV AI9 | GCTATGCAGATGCTGAAGGAGACCATC |
| 25 | AA | HIV AI9 | AMQMLKETI |
| 26 | NA | HIV AI9 | AACACCATGCTGAACACAGTGGGCGGACACCAGGCCGCTATGCAGA |
| | | 33 mer | TGCTGAAGGAGACCATCAACGAGGAGGCCGCTGAGTGGGATAGACT |
| | | | GCACCCA |
| 27 | AA | HIV AI9 | NTMLNTVGGHQAAMQMLKETINEEAAEWDRLHP |
| | | 33 mer | |
| 28 | NA | HBV | GTGTGGCTGAGCGTGATCTGGATG |
| | | VM8 | |
| 29 | AA | HBV | VWLSVIWM |
| | | VM8 | |
| 30 | NA | HBV | TCCTTCGTGCAGTGGTTTGTGGGCCTGTCTCCCACAGTGTGGCTGAG |
| | | VM8 | CGTGATCTGGATGATGTGGTACTGGGGACCTTCCGTGTACTCTATCC |
| | | 33 mer | TGAGC |
| 31 | AA | HBV | SFVQWFVGLSPTVWLSVIWMMWYWGPSVYSILS |
| | | VM8 | |
| | | 33 mer | |
| 32 | NA | MLV SF9 | TCCCCATCTTACGTGTACCACCAGTTC |
| 33 | AA | MLV SF9 | SPSYVYHQF |
| 34 | NA | MLV SF9 | GTGCTGGTGGAGCTGTGGCCTCGGGTGACCTACCACTCCCCATCTTA |
| | | 33 mer | CGTGTACCACCAGTTCGAGCGGCGCGCTAAGTACAAGAGGGAGCCC |
| | | | GTGAGC |
| 35 | AA | MLV SF9 | VLVELWPRVTYHSPSYVYHQFERRAKYKREPVS |
| | | 33 mer | |
| 36 | NA | HSV SL8 | TCTAGCATCGAGTTCGCTCGCCTG |
| 37 | AA | HSV SL8 | SSIEFARL |
| 38 | NA | HSV SL8 | CCTTCCGCTAACGCCTCCGTGGAGAGGATCAAGACCACATCTAGCAT |
| | | 33 mer | CGAGTTCGCTCGCCTGCAGTTTACATACAACCACATCCAGAGGCACG |
| | | | TGAAC |
| 39 | AA | HSV SL8 | PSANASVERIKTTSSIEFARLQFTYNHIQRHVN |
| | | 33 mer | |
| 40 | NA | RSV SI9 | TCCTACATCGGATCTATCAACAACATC |
| 41 | AA | RSV SI9 | SYIGSINNI |
| 42 | NA | RSV SI9 | GAGGAGTACGCTCTGGGCATCGTGGGAGTGCTGGAGTCCTACATCG |
| | | 33 mer | GATCTATCAACAACATCACCAAGCAGTCCGCTTGCGTGGCCATGTCT |
| | | | AAGCTG |
| 43 | AA | RSV SI9 | EEYALGIVGVLESYIGSINNITKQSACVAMSKL |
| | | 33mer | |
| 44 | NA | PADRE | GCCAAGTTTGTGGCCGCTTGGACACTGAAGGCCGCTGCC |
| 45 | AA | PADRE | AKFVAAWTLKAAA |
| 46 | NA | Furin | AGGGGCAGGAAGAGGAGATCC |
| | | NA.1 | |
| 47 | NA | Furin | AGGGGAAGAAAGCGGCGCAGC |
| | | NA.2 | |
| 48 | NA | Furin | AGGGGCAGGAAGCGGCGCAGC |
| | | NA.3 | |
| 49 | NA | Furin | CGCGGAAGGAAGAGGAGATCT |
| | | NA.4 | |
| 50 | NA | Furin | AGAGGCCGGAAGCGGCGCAGC |
| | | NA.5 | |
| 51 | NA | Furin | AGAGGCCGGAAGAGGAGAAGC |
| | | NA.6 | |
| 52 | NA | Furin | CGCGGAAGGAAGCGGCGCAGC |
| | | NA.7 | |
| 53 | NA | Furin | AGAGGCCGGAAAAGGAGAAGC |
| | | NA.8 | |
| 54 | NA | Furin | CGCGGCAGGAAGAGGAGAAGC |
| | | NA.9 | |
| 55 | NA | Furin | AGAGGCCGGAAACGGCGCTCC |
| | | NA.10 | |
| 56 | NA | Furin | CGCGGCAGGAAAAGGAGATCT |
| | | NA.11 | |
| 57 | AA | Furin | RGRKRRS |
| 58 | NA | All | ATGCTGCCAGGACAGGAGATCGACAAGGCCGATTCTTACATGCCCTA |
| | | epitopes+ | CCTGATCGACTTCGGCCTGAGCTCCAAGAGCCCATACTCTAGCGTGA |
| | | PADRE | AGAACCCCAGGGGCAGGAAGAGGAGATCCACCCTGCACGGACCCAC |
| | | | ACCTCTGCTGTACAGACTGGGCGCTGTGCAGAACGAGGTGACCCTG |
| | | | ACACACCCTATCACCAAGTACATCATGGCCTGCATGAGGGGAAGAAA |
| | | | GCGGCGCAGCCCTAACAACAACACCAGAAAGTCCATCCGGATCAGG |
| | | | AGAGGCCCAGGAAGAGCTTTCGTGACAATCGGCAAGATCGGAAACA |
| | | | TGAGGCAGGCTCACTGTAGGGGCAGGAAGCGGCGCAGCAACAACA |
| | | | ACACCAGGAAGAGAATCCGGATCCAGCGCGGCCCAGGAAGGGCTTT |
| | | | TGTGACCATTGGCAAGATTGGCAACATGAGGCAGGCCCACTGCAAC |
| | | | CGCGGAAGGAAGAGGAGATCTGAGGCTAGCACCGTGGTGCTGGTG |
| | | | GGAGCTAGGGGAACAGTGTACGGATTCTGTCTGCTGTCCAACGACG |
| | | | TGTTTAGAATCGCCGAGGATATCTCTGTGAGAGGCCGGAAGCGGCG |
| | | | CAGCCCCAGAGGCAAGCTGTCCACCCGGGGAGTGCAGATCGCTTCC |
| | | | AACGAGAACATGGACGCCATGGAGTCCTCTACACTGGAGCTGCGCTC |
| | | | TAGGTACTGGAGAGGCCGGAAGAGGAGAAGCAACACCATGCTGAA |
| | | | CACAGTGGGCGGACACCAGGCCGCTATGCAGATGCTGAAGGAGACC |
| | | | ATCAACGAGGAGGCCGCTGAGTGGGATAGACTGCACCCACGCGGAA |
| | | | GGAAGCGGCGCAGCTCCTTCGTGCAGTGGTTTGTGGGCCTGTCTCCC |
| | | | ACAGTGTGGCTGAGCGTGATCTGGATGATGTGGTACTGGGGACCTT |
| | | | CCGTGTACTCTATCCTGAGCAGAGGCCGGAAAAGGAGAAGCGTGCT |
| | | | GGTGGAGCTGTGGCCTCGGGTGACCTACCACTCCCCATCTTACGTGT |
| | | | ACCACCAGTTCGAGCGGCGCGCTAAGTACAAGAGGGAGCCCGTGAG |
| | | | CCGCGGCAGGAAGAGGAGAAGCCCTTCCGCTAACGCCTCCGTGGAG |
| | | | AGGATCAAGACCACATCTAGCATCGAGTTCGCTCGCCTGCAGTTTAC |
| | | | ATACAACCACATCCAGAGGCACGTGAACAGAGGCCGGAAACGGCGC |
| | | | TCCGAGGAGTACGCTCTGGGCATCGTGGGAGTGCTGGAGTCCTACA |
| | | | TCGGATCTATCAACAACATCACCAAGCAGTCCGCTTGCGTGGCCATG |
| | | | TCTAAGCTGCGCGGCAGGAAAAGGAGATCTGCCAAGTTTGTGGCCG |
| | | | CTTGGACACTGAAGGCCGCTGCC |
| 59 | AA | All | MLPGQEIDKADSYMPYLIDFGLSSKSPYSSVKNPRGRKRRSTLHGPTPLL |
| | | epitopes+ | YRLGAVQNEVTLTHPITKYIMACMRGRKRRSPNNNTRKSIRIRRGPGRA |
| | | PADRE | FVTIGKIGNMRQAHCRGRKRRSNNNTRKRIRIQRGPGRAFVTIGKIGN |
| | | | MRQAHCNRGRKRRSEASTVVLVGARGTVYGFCLLSNDVFRIAEDISVRG |
| | | | RKRRSPRGKLSTRGVQIASNENMDAMESSTLELRSRYWRGRKRRSNTM |
| | | | LNTVGGHQAAMQMLKETINEEAAEWDRLHPRGRKRRSSFVQWFVGL |
| | | | SPTVWLSVIWMMWYWGPSVYSILSRGRKRRSVLVELWPRVTYHSPSY |
| | | | VYHQFERRAKYKREPVSRGRKRRSPSANASVERIKTTSSIEFARLQFTYNH |
| | | | IQRHVNRGRKRRSEEYALGIVGVLESYIGSINNITKQSACVAMSKLRGRK |
| | | | RRSAKFVAAWTLKAAA |
| 60 | NA | All | ATGCTGCCAGGACAGGAGATCGACAAGGCCGATTCTTACATGCCCTA |
| | | epitopes | CCTGATCGACTTCGGCCTGAGCTCCAAGAGCCCATACTCTAGCGTGA |
| | | | AGAACCCCAGGGGCAGGAAGAGGAGATCCACCCTGCACGGACCCAC |
| | | | ACCTCTGCTGTACAGACTGGGCGCTGTGCAGAACGAGGTGACCCTG |
| | | | ACACACCCTATCACCAAGTACATCATGGCCTGCATGAGGGGAAGAAA |
| | | | GCGGCGCAGCCCTAACAACAACACCAGAAAGTCCATCCGGATCAGG |
| | | | AGAGGCCCAGGAAGAGCTTTCGTGACAATCGGCAAGATCGGAAACA |
| | | | TGAGGCAGGCTCACTGTAGGGGCAGGAAGCGGCGCAGCAACAACA |
| | | | ACACCAGGAAGAGAATCCGGATCCAGCGCGGCCCAGGAAGGGCTTT |
| | | | TGTGACCATTGGCAAGATTGGCAACATGAGGCAGGCCCACTGCAAC |
| | | | CGCGGAAGGAAGAGGAGATCTGAGGCTAGCACCGTGGTGCTGGTG |
| | | | GGAGCTAGGGGAACAGTGTACGGATTCTGTCTGCTGTCCAACGACG |
| | | | TGTTTAGAATCGCCGAGGATATCTCTGTGAGAGGCCGGAAGCGGCG |
| | | | CAGCCCCAGAGGCAAGCTGTCCACCCGGGGAGTGCAGATCGCTTCC |
| | | | AACGAGAACATGGACGCCATGGAGTCCTCTACACTGGAGCTGCGCTC |
| | | | TAGGTACTGGAGAGGCCGGAAGAGGAGAAGCAACACCATGCTGAA |
| | | | CACAGTGGGCGGACACCAGGCCGCTATGCAGATGCTGAAGGAGACC |
| | | | ATCAACGAGGAGGCCGCTGAGTGGGATAGACTGCACCCACGCGGAA |
| | | | GGAAGCGGCGCAGCTCCTTCGTGCAGTGGTTTGTGGGCCTGTCTCCC |
| | | | ACAGTGTGGCTGAGCGTGATCTGGATGATGTGGTACTGGGGACCTT |
| | | | CCGTGTACTCTATCCTGAGCAGAGGCCGGAAAAGGAGAAGCGTGCT |
| | | | GGTGGAGCTGTGGCCTCGGGTGACCTACCACTCCCCATCTTACGTGT |
| | | | ACCACCAGTTCGAGCGGCGCGCTAAGTACAAGAGGGAGCCCGTGAG |
| | | | CCGCGGCAGGAAGAGGAGAAGCCCTTCCGCTAACGCCTCCGTGGAG |
| | | | AGGATCAAGACCACATCTAGCATCGAGTTCGCTCGCCTGCAGTTTAC |
| | | | ATACAACCACATCCAGAGGCACGTGAACAGAGGCCGGAAACGGCGC |
| | | | TCCGAGGAGTACGCTCTGGGCATCGTGGGAGTGCTGGAGTCCTACA |
| | | | TCGGATCTATCAACAACATCACCAAGCAGTCCGCTTGCGTGGCCATG |
| | | | TCTAAGCTG |
| 61 | AA | All | MLPGQEIDKADSYMPYLIDFGLSSKSPYSSVKNPRGRKRRSTLHGPTPLL |
| | | epitopes | YRLGAVQNEVTLTHPITKYIMACMRGRKRRSPNNNTRKSIRIRRGPGRA |
| | | | FVTIGKIGNMRQAHCRGRKRRSNNNTRKRIRIQRGPGRAFVTIGKIGN |
| | | | MRQAHCNRGRKRRSEASTVVLVGARGTVYGFCLLSNDVFRIAEDISVRG |
| | | | RKRRSPRGKLSTRGVQIASNENMDAMESSTLELRSRYWRGRKRRSNTM |
| | | | LNTVGGHQAAMQMLKETINEEAAEWDRLHPRGRKRRSSFVQWFVGL |
| | | | SPTVWLSVIWMMWYWGPSVYSILSRGRKRRSVLVELWPRVTYHSPSY |
| | | | VYHQFERRAKYKREPVSRGRKRRSPSANASVERIKTTSSIEFARLQFTYNH |
| | | | IQRHVNRGRKRRSEEYALGIVGVLESYIGSINNITKQSACVAMSKL |
| 62 | NA | VSV PL8 | CCCTACCTGATCGACTTCGGCCTG |
| 63 | AA | VSV PL8 | PYLIDFGL |

What is claimed is:

1. A nucleic acid molecule comprising one or more nucleotide sequence encoding one or more antigen, wherein said one or more antigen comprises:
a) an established T cell epitope;
b) a series of amino acids N-terminal to said established T cell epitope derived from the endogenous protein from which the established T cell epitope originates; and
c) a series of amino acids C-terminal to said established T cell epitope derived from the endogenous protein from which the established T cell epitope originates.

2. The nucleic acid molecule of claim 1, wherein said one or more antigen is a viral antigen and wherein said established T cell epitope is an established T cell viral epitope.

3. The nucleic acid molecule of claim 2, wherein said established T cell viral epitope is derived from one or more virus selected from the group consisting of: Vesicular stomatitis virus (VSV), Hepatitis C virus (HCV), Human immunodeficiency virus 1 (HIV-1), Murine Cytomegalovirus (MCMV), Influenza A virus, Hepatitis B virus (HBV), Murine leukemia virus (MLV), Herpes simplex virus 1 (HSV-1), Herpes simplex virus 2 (HSV-2), and Respiratory syncytial virus (RSV).

4. The nucleic acid molecule of claim 3, wherein said established T cell viral epitope comprises a peptide between 8 and 10 amino acids in length.

5. The nucleic acid molecule of claim 4, wherein said established T cell viral epitope is encoded by one or more nucleotide sequence selected from the group consisting of: SEQ ID NO: 1, SEQ ID NO: 5, SEQ ID NO: 9, SEQ ID NO: 13, SEQ ID NO: 16, SEQ ID NO: 20, SEQ ID NO: 24, SEQ ID NO: 28, SEQ ID NO: 32, SEQ ID NO: 36, SEQ ID NO: 40 and SEQ ID NO: 62.

6. The nucleic acid molecule of claim 4, wherein said established T cell viral epitope comprises an amino acid sequence that is one or more selected from the group consisting of: SEQ ID NO: 2, SEQ ID NO: 6, SEQ ID NO: 10, SEQ ID NO: 17, SEQ ID NO: 21, SEQ ID NO: 25, SEQ ID NO: 29, SEQ ID NO: 33, SEQ ID NO: 37, SEQ ID NO: 41, and SEQ ID NO: 63.

7. The nucleic acid molecule of claim 1, wherein the series of amino acids N-terminal to said established T cell epitope is between 11 and 13 amino acids in length.

8. The nucleic acid molecule of claim 7, wherein the series of amino acids N-terminal to said established T cell epitope are at least 95% identical to a sequence N-terminal to the established T cell viral epitope in the endogenous protein from which the epitopes derives.

9. The nucleic acid molecule of claim 8, wherein the sequence N-terminal to the established T cell viral epitope in the endogenous protein from which the epitopes derives is directly upstream of the established T cell epitope.

10. The nucleic acid molecule of claim 1, wherein the series of amino acids C-terminal to said established T cell epitope is between 11 and 13 amino acids in length.

11. The nucleic acid molecule of claim 10, wherein the series of amino acids C-terminal to said established T cell epitope are at least 95% identical to a sequence C-terminal to the established T cell viral epitope in the endogenous protein from which the epitopes derives.

12. The nucleic acid molecule of claim 11, wherein the sequence C-terminal to the established T cell viral epitope in the endogenous protein from which the epitopes derives is directly downstream of the established T cell epitope.

13. The nucleic acid molecule of claim 2, wherein said one or more viral antigen is encoded by one or more nucleotide sequence selected from the group consisting of: SEQ ID NO: 3, SEQ ID NO: 7, SEQ ID NO: 11, SEQ ID NO: 14, SEQ ID NO: 18, SEQ ID NO: 22, SEQ ID NO: 26, SEQ ID NO: 30, SEQ ID NO: 34, SEQ ID NO: 38, and SEQ ID NO: 42.

14. The nucleic acid molecule of claim 2, wherein said one or more viral antigen comprises one or more amino acid sequence selected from the group consisting of: SEQ ID NO: 4, SEQ ID NO: 8, SEQ ID NO: 12, SEQ ID NO: 15, SEQ ID NO: 19, SEQ ID NO: 23, SEQ ID NO: 27, SEQ ID NO: 31, SEQ ID NO: 35, SEQ ID NO: 39, and SEQ ID NO: 43.

15. The nucleic acid molecule of claim 1, comprising two or more nucleotide sequences encoding two or more antigens.

16. The nucleic acid molecule of claim 15, wherein said two or more nucleotide sequences are separated by one or more nucleotide sequence encoding a furin cleavage domain.

17. The nucleic acid molecule of any one of claims 1-16, further comprising a nucleotide sequence encoding a Pan DR CD4+ helper epitope.

18. The nucleic acid molecule of claim 17, wherein the Pan DR CD4+ helper epitope comprises SEQ ID NO:45.

19. An immunogenic composition comprising the nucleic acid molecule of any one of claims 1-18.

20. A method of administering an immunogenic composition to a subject in need thereof, the method comprising administering to said subject a nucleic acid molecule comprising one or more nucleotide sequence encoding one or more antigen, wherein said one or more antigen comprises:
a) an established T cell epitope between 8 and 10 amino acids in length;
b) a series of amino acids N-terminal to said established T cell epitope derived from the endogenous protein from which the established T cell epitope originates and between 11 and 13 amino acids in length; and
c) a series of amino acids C-terminal to said established T cell epitope derived from the endogenous protein from which the established T cell epitope originates and between 11 and 13 amino acids in length.

21. A method of treating or protecting against viral infection in a subject in need thereof, the method comprising administering to said subject a nucleic acid molecule comprising one or more nucleotide sequence encoding one or more viral antigen, wherein said one or more antigen comprises:
a) an established T cell viral epitope between 8 and 10 amino acids in length;
b) a series of amino acids N-terminal to said established T cell viral epitope derived from the endogenous protein from which the established T cell viral epitope originates and between 11 and 13 amino acids in length; and
c) a series of amino acids C-terminal to said established T cell viral epitope derived from the endogenous protein from which the established T cell viral epitope originates and between 11 and 13 amino acids in length.

22. The method of claim 21, wherein said established T cell viral epitope and said endogenous protein is derived from one or more virus selected from the group consisting of: VSV, HCV, HIV-1, MCMV, Influenza A virus, HBV, MLV, HSV-1, HSV-2, or RSV.
